(19) United States
(12) Patent Application Publication
(10) Pub. No.: US 2011/0117220 A1
(43) Pub. Date: May 19, 2011

(54) PREPARATION METHOD OF PLANT EXTRACT USING HIGH PRESSURE-ENZYMATIC DECOMPOSITION TECHNIQUE AND THE COSMETIC COMPOSITION CONTAINING THE EXTRACT

(75) Inventors: Hyun Seo Kang, Yongin-si (KR); Hye Won Kim, Seoul (KR); Chan Koo Kang, Yongin-si (KR); Sung A. Cho, Anyang-si (KR); Jun Cheol Cho, Suwon-si (KR); Sang Hoon Han, Suwon-si (KR)

(73) Assignee: AMOREPACIFIC CORPORATION, Seoul (KR)

(21) Appl. No.: 12/944,775

(22) Filed: Nov. 12, 2010

(30) Foreign Application Priority Data
Nov. 13, 2009 (KR) 10-2009-0109850

(51) Int. Cl. A61K 36/82; A61K 36/00; A61K 36/899

(52) U.S. Cl. 424/729; 424/725; 424/750

(57) ABSTRACT

The present invention relates to a method of preparing a plant extract using a high-pressure enzymatic decomposition technique (HPED technique) and to a cosmetic composition containing the prepared plant extract as an active ingredient. The plant extract prepared using the high-pressure enzymatic decomposition technique developed according to the present invention contains various kinds and large amounts of effective components compared to extracts prepared using other extraction techniques, such that the effects of the effective components can be maximized.

### TECHNICAL FIELD

The present invention relates to a method of preparing a plant extract using a high-pressure enzymatic decomposition technique (HPED technique) and to a cosmetic composition containing the prepared plant extract as an active ingredient. The plant extract prepared using the high-pressure enzymatic decomposition technique developed according to the present invention contains various kinds and large amounts of effective components compared to extracts prepared using other extraction techniques, such that the effects of the effective components can be maximized.

### BACKGROUND ART

In a conventional solvent extraction technique of extracting active ingredients from a plant, a process of adding the plant to water, an organic solvent or a mixture of water and an organic solvent (e.g., ethanol, methanol, butanol, ether, ethyl acetate, chloroform or hexane) and allowing the plant in the solvent at room temperature for one day is repeated twice or more to obtain an extract. The extract is filtrated, and the filtrate is concentrated in a vacuum concentrator to obtain a first product. Water and an organic solvent are added to the first product, after which the solution is stirred at room temperature for 2 hours or more and then allowed to stand, thereby separating the solution into layers. After the layer separation, the water layer is removed, and then an organic solvent is additionally added. The above process is repeated twice or more, and the obtained extract is sufficiently washed and filtered. The filtrate is dried in a vacuum oven, thereby obtaining a desired extract. However, this solvent extraction technique has problems in that the extraction yield is low and that the organic solvent can remain, thus causing problems in terms of safety. For these reasons, a new extraction method has been required.

Plant extraction methods which were recently developed include supercritical fluid extraction (SFE). This extraction method has an advantage in that the use of supercritical fluid (e.g., liquefied carbon dioxide or liquefied propane), but it has problems in that it is expensive and that the extraction of various substances at critical temperatures cannot be guaranteed.

### DISCLOSURE OF INVENTION

Accordingly, the present inventors have conducted many studies to find an extraction technique capable of preparing a plant extract containing large amounts of various effective components and, as a result, have found that a plant extract prepared using a high-pressure enzymatic decomposition technique contains large amounts of various active ingredients, and thus has excellent antioxidant, whitening and moisturizing effects, thereby completing the present invention.

Therefore, it is an object of the present invention to provide a method of preparing a plant extract containing large amounts of various effective components using a high-pressure enzymatic decomposition technique, and a cosmetic composition containing the extract as an active ingredient.

To achieve the above object, the present invention provides a method of preparing a plant extract, which comprises a high-pressure enzymatic decomposition step of treating a raw material with an enzyme at a high pressure of 400-800 MPa.

The present invention also provides a cosmetic composition containing, as an active ingredient, the plant extract prepared using the high-pressure enzymatic decomposition technique.

### EFFECTS OF THE INVENTION

Plant extracts prepared using the high-pressure enzymatic decomposition technique according to the present invention contains various kinds and large amounts of effective components compared to extracts prepared using other extraction techniques, such that the effects of the effective components can be maximized.

A composition containing a green tea extract among the plant extracts which are provided according to the present invention shows the effects of eliminating DPPH radicals and promoting glutathione synthesis, and thus has an excellent antioxidant effect. Also, it suppresses melanin synthesis, inhibits tyrosinase activity, and thus has an excellent whitening effect. Accordingly, it can be used as an antioxidant and whitening composition. Moreover, a composition containing a bamboo extract prepared according to the method of the present invention promotes transglutaminase-1 synthesis, and thus has excellent skin barrier-enhancing and moisturizing effects. Also, because the bamboo extract-containing composition suppresses melanin synthesis, it has an excellent whitening effect. Accordingly, the bamboo extract-containing composition can be used as a skin moisturizing and whitening composition.

### BEST MODE

The present invention provides a method of preparing a plant extract, which comprises a high-pressure enzymatic decomposition step of treating a raw material with an enzyme at a high pressure of 400-800 MPa.

The present invention also provides a cosmetic composition containing, as an active ingredient, the plant extract prepared using the high-pressure enzymatic decomposition technique.

Hereinafter, the present invention will be described in further detail.

According to the present invention, a plant extract is prepared using enzymatic decomposition at high pressure, such that effective components such as trace amounts of amino acids, which were not obtainable by conventional extraction methods, can be extracted. Also, because the effective components can be extracted in large amounts, the effects of the effective components can be maximized.

The method of preparing the plant extract according to the present invention comprises a high-pressure enzymatic decomposition step of treating a raw material with an enzyme at a high pressure of 400-800 MPa. Also, the method of the present invention may further comprise a step of filtering and diluting the extract resulting from the high-pressure enzymatic decomposition step.

Each step of the method of preparing the plant extract using the high-pressure enzymatic decomposition according to the present invention will now be described.

1) High-Pressure Enzymatic Decomposition Step of Treating Raw Material with Enzyme at High Pressure of 400-800 MPa

A raw material is decomposed with an enzyme at a pressure of 400-800 MPa, and preferably 600 MPa corresponding to a sea depth of 6,000 m. At a pressure of less than 400 MPa, effective components will not be sufficiently extracted, and at a pressure of more than 800 MPa, an increase in the amount of effective components increased will be insignificant. For this reason, the enzymatic decomposition is performed in the above-specified pressure range.

The method of preparing the plant extract according to the present invention is applicable to the extraction of all plants known in the art. A specific example of a raw material which can be used in the present invention is at least one selected from the group consisting of green tea, bamboo and adlay.

An enzyme which can be used in the present invention is at least one selected from the group consisting of amylase, protease, glycosidase, lactase, sucrose and maltase.

Also, the temperature of the enzymatic decomposition is controlled at a temperature of 30˜60° C. depending on the activation temperature of the enzyme. If the enzymatic decomposition temperature is higher than 60° C., the enzyme will be broken to lose its function, and for this reason, the temperature is controlled at a temperature of 60° C. or below.

The raw material and the enzyme are mixed at a weight ratio of 100,000:1-100:1. If the mixing ratio is less than 100,000:1, the extraction of the effective components will be insufficient, and if the mixing ratio is more than 100:1, an increase in the amount of components extracted will be insignificant.

2) Step of Filtering Solution Resulting from Enzymatic Decomposition

The solution resulting from enzymatic decomposition in step 1) is filtered to remove impurities, whereby a crude plant extract can be obtained. Any conventional filtration method may be used in the present invention. For example, the solution resulting from enzymatic decomposition may be filtered through fine filter paper, thereby obtaining a crude plant extract from which impurities were removed.

3) Step of Diluting Filtered Solution

The crude plant extract filtered in step 2) is diluted in a suitable solvent in order to make the use of the extract convenient. In this step, any conventional solvent known in the art may be used. For example, water, butylene glycol or a mixed solvent thereof may be used.

In another aspect, the present invention provides a cosmetic composition containing the plant extract prepared using the high-pressure enzymatic decomposition technique.

A cosmetic composition containing a green tea extract prepared using the high-pressure enzymatic decomposition technique according to the present invention shows the effects of removing DPPH radicals and promoting glutathione synthesis, and thus has an excellent antioxidant effect. Also, it suppresses melanin synthesis and inhibits tyrosinase activity, suggesting that it has an excellent whitening effect. Thus, the composition may be used as a skin whitening and antioxidant composition.

Moreover, a composition containing a bamboo extract prepared using the high-pressure enzymatic decomposition technique according to the present invention promotes transglutaminase-1 synthesis, and thus shows excellent skin barrier-enhancing and moisturizing effects. Also, it suppresses melanin synthesis, and thus shows an excellent whitening effect. Accordingly, it may be used as a skin moisturizing, whitening and antioxidant cosmetic composition.

The plant extract of the present invention is contained in an amount of 0.000001-10 wt %, and preferably 0.001-5 wt %, based on the total weight of the composition. If the content of the plant extract is less than 0.000001 wt %, the effect thereof will be insignificant, and if the content is more than 10 wt %, an increase in the effect thereof will not be significant.

The cosmetic composition of the present invention can be formulated in various forms, including, but not limited to, skin lotion, astringent lotion, milk lotion, nourishing cream, massage cream, essence, eye cream, eye essence, cleansing cream, cleaning foam, cleansing water, pack, powder, body lotion, shampoo, rinse, body cleanser, tooth paste and oral cleaner.

### BEST MODE FOR CARRYING OUT THE INVENTION

Hereinafter, the present invention will be described in further detail with reference to examples and test examples, but the scope of the present invention is not limited only to these examples.

### Example 1

### Preparation of Green Tea Extract Using High-Pressure Enzymatic Decomposition Technique

Protease (0.1 g) was added to and mixed with green tea leaves (100 g) at a pressure of 600 MPa and a temperature of 50° C., thereby obtaining an enzymatically decomposed crude green tea extract. Then, the crude extract was filtered through filter paper to remove impurities and diluted in a solvent of water:butylene glycol (2:1, v/v) at a concentration of 1%, thereby preparing a green tea extract.

### Example 2

### Preparation of Bamboo Extract Using High-Pressure Enzymatic Decomposition Technique

Protease (0.1 g) was added to and mixed with a bamboo (100 g) at a pressure of 600 MPa and a temperature of 50° C., thereby obtaining an enzymatically decomposed crude bamboo extract. Then, the crude extract was filtered through filter paper to remove impurities and diluted in a solvent of water:butylene glycol (2:1, v/v) at a concentration of 1%, thereby preparing a bamboo extract.

### Comparative Example 1

### Preparation of Green Tea Extract Using High-Pressure Extraction Technique

Green tea leaves (100 g) were extracted at a pressure of 600 MPa, and then filtered through filter paper. Then, the filtrate was diluted in a solvent of water:butylene glycol (2:1, v/v) at a concentration of 1%, thereby obtaining a green tea extract.

### Comparative Example 2

### Preparation of Green Tea Extract Using Enzymatic Decomposition Technique

Protease (0.1 g) was added to and mixed with green tea leaves (100 g) at a temperature of 50° C., thereby obtaining an enzymatically decomposed solution. Then, the solution was filtered through filter paper and diluted in a solvent of water:butylene glycol (2:1, v/v) at a concentration of 1%, thereby preparing a green tea extract.

### Comparative Example 3

### Preparation of Green Tea Extract Using Ethanol Extraction Technique

An extraction process of adding green tea leaves (10 g) to 50 vol % ethanol (100 ml) and allowing the green tea extract at room temperature for one day was repeated twice, thereby obtaining an extract. The extract was concentrated in a vacuum concentrator, and water and ethanol were added to the concentrate. Then, the solution was stirred at room temperature for 2 hours, and then allowed to stand, so that it was separated into layers. After the layer separation, the water layer was removed, and ethanol was additionally added. This process was repeated twice, and the resulting material was sufficiently washed, filtered, and dried in a vacuum oven, thereby preparing a green tea extract.

### Comparative Example 4

### Preparation of Bamboo Extract Using Ethanol Extraction Technique

A process of adding a bamboo (10 g) to 50 vol % ethanol (100 ml) and allowing the bamboo at room temperature for one day was repeated twice, thereby obtaining an extract. The extract was filtered and concentrated in a vacuum concentrator. Water and ethanol were added to the concentrate, and the solution was stirred at room temperature for 2 hours, and then allowed to stand, so that it was separated into layers. After the layer separation, the water layer was removed, and ethanol was additionally added. This process was repeated twice, and the resulting material was sufficiently washed, filtered, and then dried in a vacuum oven, thereby preparing a bamboo extract.

### Test Example 1

### Comparison of Amino Acid Content Between Green Tea Extracts Obtained Using High-Pressure Enzymatic Decomposition Technique of the Present Invention and Conventional Extraction Technique

The contents of amino acids in the green tea extracts of Example 1 and Comparative Examples 1 to 3 were analyzed using the following OPA method. The results of the analysis are shown in Table 1 below.

<Amino Acid Analysis (OPA Method)>

1) HPLC Conditions
column: zorbax column (for amino acid analysis)
mobile phases:

A=1.36 g sodium acetate trihydrate+100 μl triethylamine→adjusted to a volume of 500 ml→pH 7.2 (adjusted with acetic acid)→1.5 ml THF

B=1.36 g sodium acetate trihydrate/100 ml H₂O→pH 7.2+200 ml methanol+200 ml ACN
flow rate: 0.5 ml/min
injection: injection program for online derivatization*
detector: 338 nm
gradient: online program*

2) Reagent Preparation
amino acid standard solution: 10 mg of each of reagents, including aspartic acid, glutaminic acid, proline, glycine, alanine and valine, and 100 ml H₂O
OPA reagent: supplied from HP Co.; storage period: 6 months; ampoule form
borate buffer: supplied in a unit of 100 ml; required for development of amino acids

TABLE 1

| | Green tea | Green tea | | Green tea |
|---|---|---|---|---|
| | extract | extract | Green tea | extract |
| | prepared | prepared | extract | prepared |
| | by high- | by high- | prepared by | by |
| | pressure | pressure | enzymatic | ethanol |
| | enzymatic | extraction | decomposition | extraction |
| | decomposition | technique | technique | technique |
| | technique | (Comp. | (Comp. Ex. | (Comp. |
| | (Ex. 1) | Ex. 1) | 2) | Ex. 3) |
| Aspartic | 0.558 | 0.518 | 0.809 | 0.562 |
| acid | | | | |
| Glutaminic | 0.747 | 0.668 | 1.006 | 0.694 |
| acid | | | | |
| Proline | 0.061 | 0.024 | less than | less than |
| | | | 0.018 | 0.017 |
| Glycine | 0.062 | 0.035 | less than | less than |
| | | | 0.005 | 0.005 |
| Alanine | 0.347 | 0.238 | 0.227 | 0.143 |
| Valine | 0.471 | 0.269 | 0.050 | 0.033 |
| Methionine | 0.073 | 0.053 | less than | less than |
| | | | 0.032 | 0.032 |
| Isoleucine | 0.273 | 0.140 | 0.052 | 0.032 |
| leucine | 0.985 | 0.491 | less than | less than |
| | | | 0.046 | 0.046 |
| Tyrosine | 0.199 | 0.132 | less than | less than |
| | | | 0.052 | 0.052 |
| Phenylalanine | 0.501 | 0.262 | less than | less than |
| | | | 0.031 | 0.031 |
| Histidine | 0.458 | 0.329 | 0.231 | 0.235 |
| Lysine | 0.401 | 0.208 | 0.038 | 0.023 |
| Arginine | 0.559 | 0.425 | 0.491 | 0.337 |
| Sum | 5.695 | 3.792 | 2.904 | 2.059 |
| Increasing | 277 | 184 | 141 | 100 |
| rate (%) | | | | |

As can be seen in Table 1 above, the total contents of amino acids in the green tea extracts prepared using the high-pressure extraction technique and the enzymatic decomposition technique (Comparative Examples 1 and 2) increased by 184% and 141%, respectively, compared to 100% of the total amino acid content of the green tea extract obtained using the ethanol extraction technique (Comparative Example 3), whereas the total amino acid content of the green tea extract obtained using the high-pressure enzymatic decomposition technique of the present invention (Example 1) significantly increased by 277%.

Thus, the plant extract prepared using the high-pressure enzymatic decomposition technique of the present invention could contain effective components in amounts larger than those in extracts obtained using other conventional extraction methods.

### Test Example 2

### Effect on DPPH (Diphenylpicryl Hyrazyl) Radical Elimination

In order to measure the antioxidant effect of the plant extract prepared using the high-pressure enzymatic decomposition technique, a DPPH radical elimination effect was compared between the green tea extract prepared using the high-pressure enzymatic decomposition technique of the present invention (Example 1), the green tea extract prepared using the solvent extraction technique (Comparative Example 3), and Baicalin, a known antioxidant substance.

To measure the antioxidant effects of these extracts, a method of evaluating antioxidant activity based on the change in absorbance caused by the reduction of the organic radical DPPH (1,1-diphenyl-2-picrylhydrazyl) was used. The decrease in absorbance caused by the inhibition of oxidation of DPPH compared to the control was measured, and the concentration (IC₅₀) at which the absorbance was 50% of the control was defined as the effective antioxidant concentration. A lower IC₅₀ value indicates a higher DPPH removal effect, indicating higher antioxidant activity.

Specifically, 10 μl of each of the extracts of Example 1 and Comparative Example 3 and Baicalin was added to 100 μM of a solution of DPPH in ethanol to prepare a reaction solution. The reaction solution was allowed to react at 37° C. for 30 minutes, and then measured for absorbance at 540 nm. The results of the measurement are shown in Table 2 below.

TABLE 2

| | IC₅₀ (mg/ml) |
|---|---|
| Example 1 | 3.4 |
| Comparative Example 3 | 6.7 |
| Baicalin | 3.1 |

As can be seen in Table 2 above, the antioxidant activity of the green tea extract of Example 1 prepared using the high-pressure enzymatic decomposition technique of the present invention was about two times higher than that of the green tea extract of Comparative Example 3 prepared using the ethanol extraction technique and was similar to that of the typical antioxidant Baicalin.

### Test Example 3

### Effect on Promotion of Glutathione Synthesis

In order to measure the antioxidant effect of the plant extract prepared using the high-pressure enzymatic decomposition technique, a glutathione synthesis-promoting effect was compared between the green tea extract of Example 1 prepared using the high-pressure enzymatic decomposition technique, the green tea extract of Comparative Example 3 prepared using the solvent extraction technique, and Rose Myrtle, a known antioxidant substance. Glutathione is a typical antioxidant contained in the human body and has the effect of suppressing reactive oxygen species. Thus, the promotion of glutathione synthesis can suppress reactive oxygen species, thereby preventing skin aging and making the skin healthy.

Specifically, 3×10⁴ fibroblast cells were added to each well of a 24-well plate and then cultured 37° C. for 12 hours. The cultured cells were treated for 24 hours with each of the green tea extract prepared using the high-pressure enzymatic decomposition technique and the green tea extract prepared using the ethanol extraction technique. As a positive control, Rose Myrtle was used. 0.9% Triton X-100 was added to the cell culture which was then allowed to react at 37° C. for 30 minutes. The cell lysate was collected and centrifuged at 2,000 rpm for 20 minutes, and the supernatant was transferred into a fresh tube. A 1/10 volume of 1M 2-vinylpyridone was added to the cell lysate, which was then allowed to react at room temperature for 1 hour. This reaction is a step of removing reduction-type glutathione, and where reduction-type glutathione was not removed, the cell lysate was subjected to the next step without performing this reaction. The same volume of 10% metaphosphoric acid was added to the cell lysate, which was then allowed to stand at room temperature for 5 minutes. Then, the lysate was centrifuged at 12,000 rpm for 2 minute, and the supernatant was collected. A ⅕ volume of 4 M triethanolamine was added to the supernatant, thereby preparing samples for quantifying oxidation-type/reduction-type glutathione. 50 μl of the sample, which was treated or not treated with 2-vinylpyridone was added to a 96-well microtiter plate, and 50 μl of G enzyme (1.28 mU/μl glutathione reductase) was added thereto, and then 100 μl of G buffer mixture (2 mM NADPH, 20 mM DTNB, 0.4M MES, 2 mM EDTA, 0.1M sodium phosphate, pH 6.0) was added. The resulting mixture was allowed to react at room temperature for 10 minutes, and the absorbance at 405 nm was measured. The results of the measurement were compared with the absorbance values obtained for the case in which the sample was not contained, thereby calculating the rate of promotion of glutathione synthesis. The results of the calculation are shown in Table 3 below.

TABLE 3

| | Concentration | Promotion rate of |
|---|---|---|
| | (mg/ml) | glutathione synthesis (%) |
| Example 1 | 100 | 148.0 ± 4.5 |
| | 200 | 181.3 ± 1.7 |
| Comp. Ex. 3 | 100 | 89.3 ± 2.3 |
| Rose Myrtle | 100 | 109.1 ± 1.2 |

As can be seen in Table 3 above, the Rose Myrtle extract known to promote glutathione synthesis showed a promotion rate of glutathione synthesis of 109%, whereas the green tea extract of Example 1 prepared using the high-pressure enzymatic decomposition technique showed a promotion rate of glutathione synthesis of 148% at the same concentration. Also, the green tea extract prepared using the high-pressure enzymatic decomposition technique of the present invention showed a strong effect of promoting glutathione synthesis, compared to the green tea extract of Comparative Example 3 prepared using the ethanol extraction method.

### Test Example 4

### Inhibition of Melanin Production

In order to measure the skin whitening effect of the plant extract prepared using the high-pressure enzymatic decomposition technique, melanin production inhibitory ability was compared between the green tea and bamboo extracts of Examples 1 and 2 prepared using the high-pressure enzymatic decomposition technique of the present invention, the green tea and bamboo extracts of Comparative Examples 3 and 4 prepared using the solvent extraction technique, and kojic acid, a typical whitening functional component.

Specifically, human melanoma HM3KO cells (Y. Funasaka, Department of dermatology, Kobe university school of medicine, 5-1 Kusunoki-cho 7-chrome, Chuo-ku, Kobe 650, Japan) were cultured in 10% FBS-containing MEM (Minimum Essential Medium) under conditions of 37° C. and 5% CO₂. The cultured cells were seeded into 75 flasks at a density of 3×10⁵ cells per flask and allowed to stand overnight until the cells adhered to the flask wall. After the cells were confirmed to adhere to the flask wall, the medium was replaced with a fresh medium containing 10 ppm of each of the test samples. As a control, DMSO-containing medium was used. While the medium was replaced with a sample-containing fresh medium at intervals of 2-3 days, the cells were cultured until the flask was filled with the cells. The cell culture was collected, washed with PBS and dissolved in 1N sodium hydroxide, and the absorbance at 500 nm was measured. Based on the results of the measurement, melanin production inhibitory rate was calculated according to the following equation 1, and the results of the calculation are shown in Table 4 below.

Melanin production inhibitory rate (%)=100×[(absorbance of each sample)/(absorbance of control)×100] [Equation 1]

TABLE 4

| | Concentration | Melanin production |
|---|---|---|
| | (mg/ml) | inhibitory rate (%) |
| Example 1 | 200 | 39.3 ± 1.2 |
| Example 2 | 200 | 28.4 ± 1.2 |
| Comp. Ex. 3 | 200 | No effect |
| Comp. Ex. 4 | 200 | No effect |
| Kojic acid | 200 | 48.9 ± 1.2 |

As can be seen in Table 4 above, the green tea extract of Example 1 prepared using the high-pressure enzymatic decomposition technique of the present invention had a melanin synthesis inhibitory ability corresponding to about 80% of the melanin synthesis inhibitory ability of kojic acid, and the bamboo extract of Example 2 of the present invention had a melanin synthesis inhibitory ability corresponding to about 60% of that of kojic acid. However, the extracts of Comparative Examples 3 and 4 prepared using the ethanol extraction technique had no melanin synthesis inhibitory ability.

### Test Example 5

### Effect on Inhibition of Tyrosinase Synthesis

In order to measure the skin whitening effect of the plant extract prepared using the high-pressure enzymatic decomposition technique, a tyrosinase activity inhibitory effect was compared between the green tea extract of Example 1 prepared using the high-pressure enzymatic decomposition technique of the present invention, the green tea extract of Comparative Example 3 prepared using the solvent extraction technique, and vitamin C, a typical whitening functional component. Vitamin C is a component effective for skin whitening which inhibits tyrosinase activity.

The tyrosinase activity inhibitory effect was measured using the method of Vanni at al (A. Vanni, Annali Di Chimica, 80, p35, 1990). Specifically, 1.0 ml of 0.1M potassium phosphate buffer (pH 6.8), 1.0 ml of 0.3 mg/ml tyrosine aqueous solution and 0.1 ml of 1,250 units/ml tyrosinase (SIGMAT-7755) were mixed with each other, and 0.2 ml of each sample solution was added thereto at a concentration of 200 mg/ml. Then, the mixture was subjected to an enzymatic reaction at 37° C., for 10 minutes. The absorbance of the reaction solution was measured at 480 nm, and based on the results of the measurement, the tyrosinase activity inhibitory rate (%) of each sample was calculated according to the following equation 2. The results of the calculation are shown in Table 5 below.

Tyrosinase activity inhibitory rate (%)=A−B/A×100 [Equation 2]

wherein A: absorbance at 480 nm of a reaction solution to which the sample was not added; and B: absorbance at 480 nm of a reaction solution to which the sample was added.

TABLE 5

| | Concentration | Tyrosinase activity |
|---|---|---|
| | (mg/ml) | inhibitory rate (%) |
| Example 1 | 200 | 21.1 |
| Comp. Ex. 3 | 200 | No effect |
| Vitamin C | 200 | 20 |

As can be seen in Table 5 above, the green tea extract of Comparative Example 3 prepared using the ethanol extraction technique had no tyrosinase activity inhibitory effect, whereas the green tea extract of Example 1 prepared using the high-pressure enzymatic decomposition technique of the present invention showed a tyrosinase activity inhibitory effect similar to or slightly higher than that of vitamin C.

### Test Example 6

### Effect on Promotion of Transglutaminase-1 Synthesis

In order to measure the skin moisturizing effect of the plant extract prepared using the high-pressure enzymatic decomposition technique, an effect on the promotion of transglutaminase-1 synthesis was compared between the bamboo extract of Example 2 prepared using the high-pressure enzymatic decomposition technique of the present invention, the bamboo extract of Comparative Example 4 prepared using the solvent extraction technique, and calcium chloride, a typical component that promotes transglutaminase-1 synthesis.

Because the synthesis of transglutaminase-1 is essential for the formation and maintenance of the horny layer, the effect of promoting the synthesis of transglutaminase-1 can be considered to enhance the skin barrier and increase the skin moisturizing effect.

Specifically, human skin cells were added to each well of a 96-well plate at a density of 5×10⁴ cells/well and allowed to adhere to each well for 24 hours. The adhered cells were treated with each of the test materials, and after 2 days, the medium was removed, and the cells were stored in a refrigerator at −20° C. The treated cells were disrupted by subjecting the cells twice to freeze-thawing, and then treated with a mixture of acetone:ethanol (1:1, v/v) stored at −20° C. Then, the cells were allowed to stand at 4° C. for 30 minutes so as to be immobilized. Then, the cells were allowed to stand at room temperature to evaporate the organic solvent. Then, the cells were blocked with 1% bovine serum albumin and incubated with transglutaminase antibody (primary antibody) and HRP anti-mouse antibody (secondary antibody), and OPD (o-phennyldiamine) was added to develop the color of the cells. The expression level of transglutaminase in the cells was determined by measuring the absorbance at 490 nm, and the correction of the measurement was carried out by measuring the background at 630 nm. The absorbance values of the treated cells were compared with the absorbance value of an untreated control group, thereby calculating the rate of promotion of transglutaminase-1 synthesis, and the results of the calculation are shown in Table 6 below.

TABLE 6

| | | | Promotion rate of |
|---|---|---|---|
| | Concentration | | transglutaminase-1 |
| | (mg/ml) | | synthesis (%) |
| Example 2 | 12.5 | | 137.0 ± 1.9 |
| | 50 | | 161.0 ± 4.0 |
| Comp. Ex. 4 | 12.5 | | No effect |
| | 50 | | No effect |
| Calcium | 1.5 | mM | 169.9 ± 6.4 |
| chloride | | | |

As can be seen in Table 6 above, the bamboo extract of Example 2 prepared using the high-pressure enzymatic decomposition technique of the present invention had the effect of promoting the synthesis of transglutaminase-1, unlike the bamboo extract of Comparative Example 4 prepared using the solvent extraction technique. Also, it was found that, when the bamboo extract of Example 2 was used at a concentration of 50 mg/ml, it had a transglutaminase-1 synthesis-promoting effect corresponding to about 95% of the effect of 1.5 mM calcium chloride, a typical component that promotes the synthesis of transglutaminase-1.

1. A method of preparing a plant extract, which comprises a high-pressure enzymatic decomposition step of treating a raw material with an enzyme at a high pressure of 400-800 MPa.

2. The method of claim 1, wherein the raw material is at least one selected from the group consisting of green tea, bamboo and adlay.

3. The method of claim 1, wherein the enzyme is at least one selected from the group consisting of amylase, protease, glycosidase, lactase, sucrose and maltase.

4. The method of claim 1, wherein the raw material and the enzyme are mixed at a weight ratio of 100,000:1-100:1.

5. The method of claim 1, wherein the enzymatic decomposition is performed at a temperature of 30˜60° C.

6. The method of claim 1, which further comprises a step of filtering and diluting the extract resulting from the high-pressure enzymatic decomposition step.

7. A cosmetic composition which contains, as an active ingredient, the plant extract prepared by the method of claim 1.

8. The cosmetic composition of claim 7, wherein the plant extract are contained in an amount of 0.000001-10 wt % based on the total weight of the composition.

9. A cosmetic composition for antioxidant, whitening and moisturizing which contains, as an active ingredient, a green tea or bamboo extract prepared by the method of claim 1.

10. The cosmetic composition of claim 9, wherein the green tea or bamboo extract are contained in an amount of 0.000001-10 wt % based on the total weight of the composition.
